(19) United States
(12) Patent Application Publication
(10) Pub. No.: US 2023/0317282 A1
(43) Pub. Date: Oct. 5, 2023

(54) PRE-DIAGNOSIS SYSTEM AND METHOD PROVIDING GUIDANCE AS TO A CORRECT MEDICAL AND DIAGNOSIS

(71) Applicant: Fulian Precision Electronics (Tianjin) Co., LTD., Tianjin (CN)

(72) Inventor: SHAO-HSUAN FAN, New Taipei (TW)

(21) Appl. No.: 17/893,406

(22) Filed: Aug. 23, 2022

(30) Foreign Application Priority Data
Mar. 29, 2022 (CN) 202210323855.2

(51) Int. Cl. G16H 50/20; G06F 3/14

(52) U.S. Cl. CPC G16H 50/20; G06F 3/14

(57) ABSTRACT

In the medical field, a pre-diagnosis system providing guidance and prompting to a doctor before examining a patient and making a diagnosis includes a user interface unit, an AI interpretation unit, a data unit, and a management unit. The user interface unit establishes a task of interpretation and uploads a first information according to a visit by a patient. The AI interpretation unit generates an interpretation. The data unit obtains a second information from the first information and updates the second information. The management unit obtains an updated second information and the interpretation and sets visiting state of the patient according to the second information and the interpretation. The efficiency and accuracy of a diagnosis is improved.

### TECHNICAL FIELD

The present disclosure relates to the technical field of medical diagnosis, in particular to a pre-diagnosis system and method.

### BACKGROUND

When a patient is seen in a hospital, a doctor will often rely on professional knowledge and work experience to diagnose and refer to information such as the vital signs in the medical information system to judge whether the patient has related diseases. Such consultation processes may be more time consuming. In addition, in the process of querying relevant vital signs through the medical information system, diagnostic errors may occur due to various human factors, such as input errors.

Therefore, improvement is desired.

### BRIEF DESCRIPTION OF THE DRAWINGS

FIG. 1 is a schematic diagram of a pre-diagnosis system according to an embodiment of the present disclosure.

FIG. 2 is a schematic diagram formed by a database and an AI interpretation unit according to an embodiment of the present disclosure.

FIG. 3 is an architecture diagram of the pre-diagnosis system based on the system of FIG. 1.

FIG. 4 is a flowchart of a pre-diagnosis method according to an embodiment of the present disclosure.

### DETAILED DESCRIPTION

The technical solutions in the embodiments of the present disclosure will be described in conjunction with the accompanying drawings in the embodiments of the present disclosure. Obviously, the described embodiments are part of the embodiments of the present disclosure, not all of them. Based on the embodiments of the present disclosure, all other embodiments obtained by those of ordinary skill in the art without creative work shall fall within the protection scope of the present disclosure. The terms used in the description of the present disclosure herein are only for the purpose of describing specific embodiments and are not intended to limit the present disclosure.

In the embodiment of the present disclosure, “at least one” refers to one or more, and multiple refers to two or more. Unless otherwise defined, all technical and scientific terms used herein have the same meanings as those commonly understood by those skilled in the technical field in the present disclosure. The terms used in the specification of the present disclosure are only for the purpose of describing specific embodiments and are not intended to limit the present disclosure.

In the embodiment of the present disclosure, “first”, “second” and other words are only used for the purpose of distinguishing description and cannot be understood as indicating or implying relative importance, or as indicating or implying order. The features defined as “first” and “second” may include one or more of the features explicitly or implicitly. In the description of the embodiments of the present disclosure, the words “exemplary” or “for example” are used as examples or explanations. Any embodiment or design described as “exemplary” or “for example” in the embodiments of the present disclosure shall not be interpreted as more preferred or advantageous than other embodiments or designs.

The present disclosure provides a pre-diagnosis system, which can automatically obtain information as to vital signs, and artificial intelligence (AI) technology is used to interpret the patient's symptoms, and the patient's visiting state is managed and set according to an interpretation, which improves the efficiency and accuracy of viewing and of medical conclusions drawn in respect of a patient.

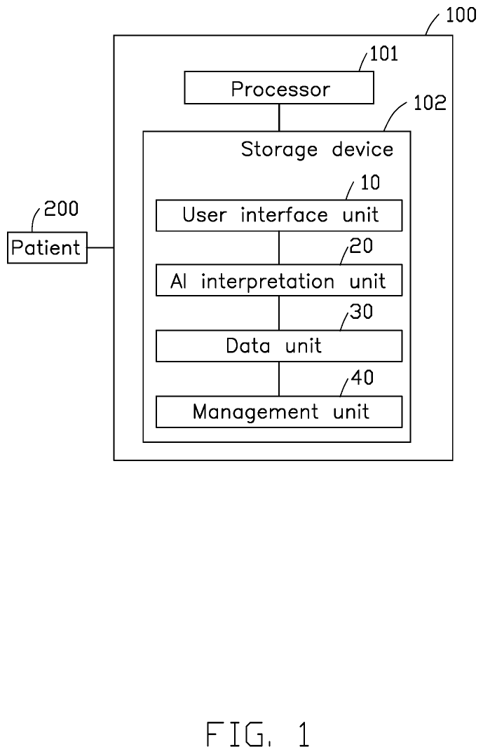

FIG. 1 illustrates a pre-diagnosis system 100 in accordance with an embodiment of the present disclosure.

The pre-diagnosis system 100 includes a processor 101 and a storage device 102. The storage device 102 can be used to store the program segment. The processor 101 operates or executes the program segment stored in the storage device 102 and calls up or recalls data stored in the storage device 102, and implements various functions of the pre-diagnosis system 100. The storage device 102 may include a plurality of functional modules composed of program code segments. For example, in the embodiment, the storage device 102 includes a user interface unit 10, an AI interpretation unit 20, a data unit 30, and a management unit 40. The program code of each program segment stored in the storage device 102 can be executed by the processor 101 of the pre-diagnosis system 100 to perform the pre-diagnosis function. In other embodiment, the units 10-40 may also be a program instruction or firmware that is embedded in the processor 101. The processor 101 is used to execute a plurality of units (e.g., the user interface unit 10, the AI interpretation unit 20, the data unit 30, and the management unit 40 shown in FIG. 1) and other applications.

The storage device 102 can be any type of non-transitory computer-readable storage medium or other computer storage device, such as a hard disk drive, a compact disc, a digital video disc, a tape drive, a storage card (e.g., a memory stick, a smart media card, a compact flash card), or other suitable storage medium, for example. The processor 101 may be a central processing unit (CPU), or may be other general purpose processor, a digital signal processor (DSP), an application specific integrated circuit (ASIC), a Field-Programmable gate array (FPGA) or other programmable logic device, a transistor logic device, or a discrete hardware component. The processor 101 is a control center of the pre-diagnosis system 100.

The user interface unit 10 is used to establish a task of interpretation.

In some embodiments, the user interface unit 10 can be a network page of a communication device (such as a computer, tablet computer, laptop computer, mobile phone), and the user can establish the task through the user interface unit 10. Specifically, the interpretation task is a task as to visits by a patient 200, created for the AI interpretation unit 20 according to the user's appointment visit behavior (such as online appointment, hospital manual appointment).

In some embodiments, the user interface unit 10 can be used to upload the first information of a visit by the patient 200 to the data unit 30 for storage when the user makes an appointment to visit. It can be understood that the first information is the information of the patient 200 needed for a visit, such as the patient's ID number, age, vital signs information, medical records, etc.

In some embodiments, the user interface unit 10 is also used to set the first preset time, such as the waiting time and time limit of the patient 200. If the patient 200 is not seen within the first preset time, the first information of the patient 200 will expire.

The AI interpretation unit 20 is connected to the user interface unit 10. The AI interpretation unit 20 is used to receive and execute the interpretation task established by the user interface unit 10, and then generate the interpretation. In some embodiments, the AI interpretation unit 20 includes a plurality of AI model programs and a plurality of interpretation modules.

The AI interpretation unit 20 is used to obtain the first information of the patient 200 and then to select the corresponding AI model program according to the first information of the patient and assign the interpretation task to the corresponding interpretation module according to the selected AI model program. The interpretation module performs the tasks assigned by the AI model program and generates interpretations, such as determining whether the patient 200 has or may have a disease, so as to perform the task. It is understandable that different interpretation modules can interpret different diseases according to the symptoms of patients.

In some embodiments, the AI model program can code the program preset in the AI interpretation unit 20. The AI interpretation unit 20 can set up the AI model programs according to the type of disease and the related symptoms of the disease. Therefore, the AI interpretation unit 20 can run the corresponding AI model program according to the relevant symptoms of the patient, and then the type of the patient and his/her condition can be obtained. When the AI interpretation unit 20 reads a new interpretation task, the AI interpretation unit 20 can further determine the symptoms of the patient according to the first information of the patient, and then select the corresponding AI model program to assign the interpretation task according to the symptoms of the patient.

For example, when the AI interpretation unit 20 determines that the vital characteristic of the patient clearly points to a type of disease, it selects an independent AI model program to assign the interpretation task. When the vital characteristic information of the patient can point to multiple disorders or diseases, the interrelated AI model program is selected to assign the interpretation task.

When the AI model program selected for the interpretation task executed by the AI interpretation unit 20 is an independent program, the AI model program assigns the interpretation task to the corresponding interpretation module, and the interpretation module independently executes the corresponding interpretation task. When the AI model program selected for the interpretation task performed by the AI interpretation unit 20 is an interrelated program, the AI model program assigns the interpretation task to several interpretation modules, and each interpretation module executes its own interpretation.

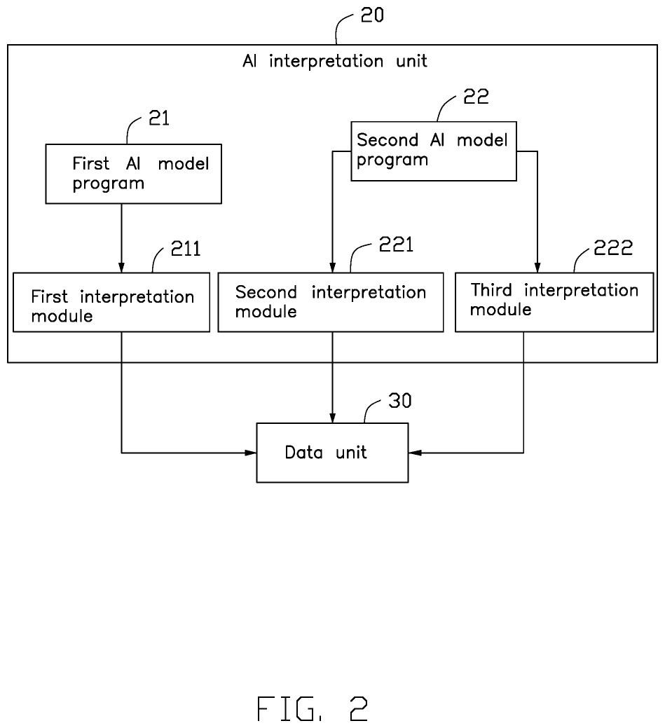

FIG. 2 shows the AI interpretation unit 20 as including two AI model programs and three interpretation modules, such as the first AI model program 21, the second AI model program 22, the first interpretation module 211, the second interpretation module 221, and the third interpretation module 222 as examples.

In the embodiment, the first AI model program 21 is used to assign the first interpretation module 211 to independently perform the corresponding interpretation. The second AI model program 22 is used to assign the second interpretation module 221 and the third interpretation module 222 to perform the corresponding interpretations. The first interpretation module 211, the second interpretation module 221, and the third interpretation module 222 are used to perform the assigned tasks of interpretation and generate results.

When the first interpretation module 211 executes and completes the corresponding interpretation task, the first interpretation result is generated. The second interpretation module 221 generates a second interpretation result after performing the corresponding interpretation task. When the second interpretation module 221 analyzes the patient as having a disease, the third interpretation module 222 continues the next interpretation task, and the third interpretation module 222 generates a third interpretation. If the second interpretation module 221 does not determine that the patient has a certain disease, the third interpretation module 222 will no longer perform its interpretation task.

As shown in FIG. 2, the interpretation results generated by the AI interpretation unit 20, such as the first interpretation result, the second interpretation result, and/or the third interpretation result, will be transmitted to the data unit 30.

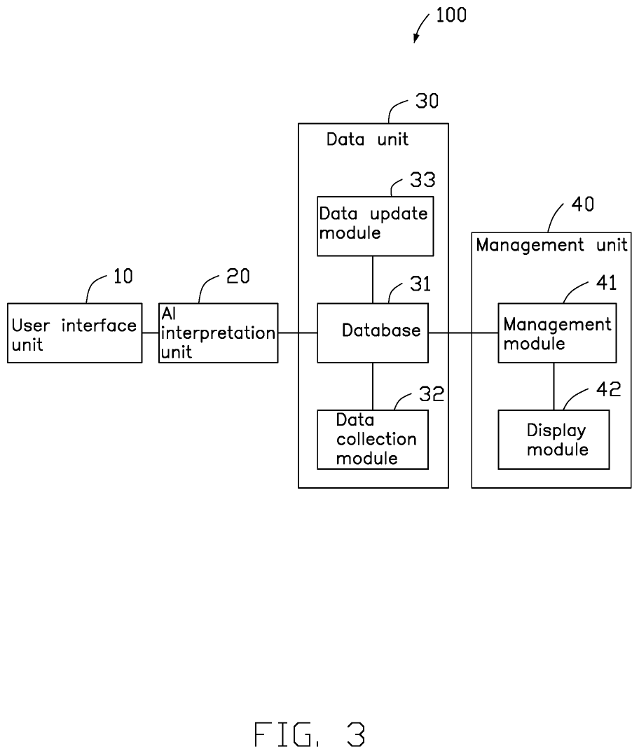

FIG. 3 shows that the data unit 30 includes a database 31, a data collection module 32, and a data update module 33.

The database 31 is used to store the first information and the interpretation results obtained by the AI interpretation unit 20, such as the first interpretation result, the second interpretation result, and/or the third interpretation result.

In some embodiments, when the time limit for visiting by the patient has not exceeded the first preset time, the database 31 sets the first information of the patient as the second information. The second information is effective visiting information. When the patient has not been seen within the first preset time, the database 31 sets the first information of the patient as the third information, and the third information is invalid.

The data collection module 32 is connected to the database 31. The data collection module 32 is used to obtain the second information in the database 31.

The data update module 33 is connected to the database 31. The data update module 33 is used to update the second information in the database 31. In some embodiments, the data update module 33 can be the medical information system of the hospital. The data update module 33 can extract the inspection data of all inspection equipment in the hospital and update the patient's inspection information (for example, with the X-ray image of the patient), such as the data update module 33 updates the second information. The present disclosure can obtain the latest life and physical information of the patient in real time by setting the data update module 33. After updating the second information, the data update module 33 stores the updated second information in the database 31.

In some embodiments, the pre-diagnosis system 100 can set a second preset time through the user interface unit 10. The second preset time is the interval between each update of the second information by the data update module 33.

The management unit 40 is connected to the database 31 of the data unit 30.

The management unit 40 is used to obtain the interpretation result obtained by the AI interpretation unit 20 and the second information updated by the data update module 33 from the database 31 and manage and set the visiting state of the patient according to the interpretation result and the updated second information. For example, the management unit 40 sets the order of patient visits according to the severity and urgency of the interpretation results. At the same time, the management unit 40 displays the visiting state of the patient to prompt the doctor and the patient.

As shown in FIG. 3, the management unit 40 includes a management module 41 and a display module 42. The management module 41 is connected to the database 31. The display module 42 is connected to the management module 41.

In some embodiments, the management module 41 can determine the severity and urgency of the symptoms of the patients according to the interpretation results and the second information, and then set the viewing sequence of the patients according to the severity and urgency of the symptoms of the patients.

In some embodiments, the management module 41 can set a program code to determine the severity and urgency of the symptoms of the patient, and preset the determination standard, such as the first standard, in the program code. For example, when the management module 41 determines that the severity and urgency of the symptoms of the patient meet the first standard, it indicates that the patient needs to be seen as soon as possible, and the order of the visit in a queue needs to be advanced. In this way, the present disclosure can determine whether the severity and urgency of the symptoms of the patient meet the first standard, that is, whether the treatment sequence needs to be advanced, by running the program code in the management module 41.

When the management module 41 determines that the severity and urgency of the symptoms of the patient meet the first standard according to the interpretation results and the second information, the management module 41 sets the visiting state of the patient to the first state. When the management module 41 determines that the severity and urgency of the symptoms of the patient do not meet the first standard according to the interpretation results and the second information, the management module 41 sets the visiting state of the patient to the second state.

The display module 42 is used to display the signals according to the settings of the management module 41 to remind doctors and patients as to a visit. When the management module 41 sets the visiting state of the patient as the first state, the display module 42 displays the first displaying information. When the management module 41 sets the visiting state of the patient as the second state, the display module 42 displays the second displaying information. For example, the display module 42 may be an LED lamp. When the management module 41 sets the visiting state of the patient to the first state, the display module 42 emits a red light. When the management module 41 sets the visiting state of the patient to the first state, the display module 42 emits a yellow light.

In some embodiments, the management unit 40 can also send the visiting state set by the management module 41 to the mobile terminal (not shown in the figure) through wireless signals (such as BLUETOOTH, infrared, mobile network), and then the mobile terminal can display the visiting state, and doctors and patients can view the visiting state through the mobile terminal. It is understandable that mobile terminals can be, but are not limited to, computers, tablets, laptops, mobile phones and other terminals, which are not specifically limited here.

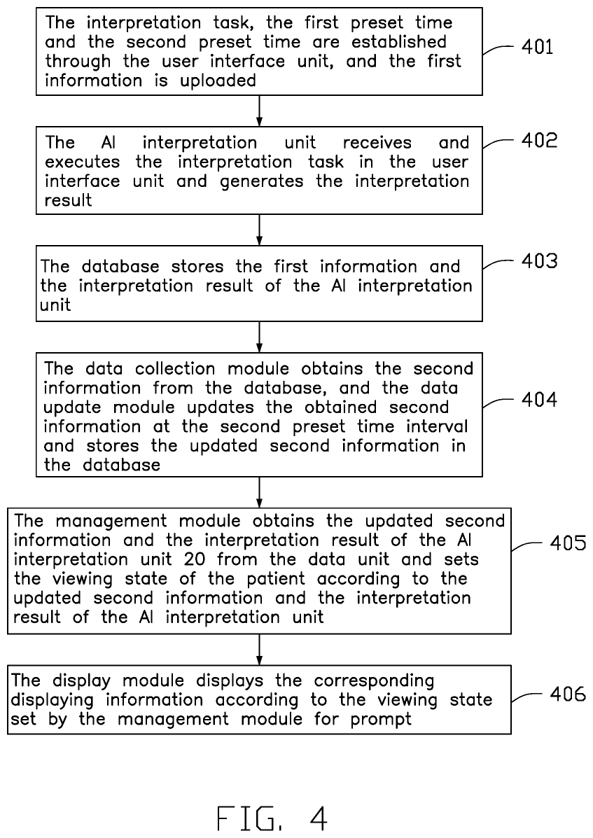

FIG. 4 is a flowchart depicting an embodiment of a pre-diagnosis method. The pre-diagnosis method is applied in the pre-diagnosis system 100.

Each block shown in FIG. 4 represents one or more processes, methods, or subroutines, carried out in the example method. Furthermore, the illustrated order of blocks is illustrative only and the order of the blocks can change. Additional blocks can be added or fewer blocks may be utilized, without departing from the present disclosure. The example method can begin at block 401.

At block 401, the interpretation task, the first preset time and the second preset time are established through the user interface unit 10, and the first information is uploaded.

At block 402, the AI interpretation unit 20 receives and executes the interpretation task in the user interface unit 10 and generates the interpretation result.

At block 403, the database 31 stores the first information and the interpretation result of the AI interpretation unit 20.

In some embodiments, when the time limit for visiting by the patient has not exceeded the first preset time, the database 31 sets the first information of the patient as the second information, and the second information is valid visiting information.

At block 404, the data collection module 32 obtains the second information from the database 31, and the data update module 33 updates the obtained second information at the second preset time interval and stores the updated second information in the database 31.

At block 405, the management module 41 obtains the updated second information and the interpretation result of the AI interpretation unit 20 from the data unit 30 and sets the visiting state of the patient according to the updated second information and the interpretation result of the AI interpretation unit 20.

At block 406, the display module 42 displays the displaying information according to the visiting state set by the management module 41 for prompting.

The pre-diagnosis system 100 of the present disclosure can preset the AI model program according to the type of disease and the relevant symptoms of the disease, and then select the AI model program according to the visiting information of the patient, assign the interpretation task to the AI interpretation module, and analyze information as to the vital signs of the patient. The present disclosure can pre-diagnose the disease of the patient, manage and set the visiting state according to the pre-diagnosis results, and display the set visiting state to prompt, which improves the efficiency of visiting. The pre-diagnosis system 100 also adopts the data update module 33 to update the vital signs information of the patient, which improves the accuracy of diagnosis.

Those of ordinary skill in the art should realize that the above embodiments are only used to illustrate the present disclosure, but not to limit the present disclosure. As long as they are within the essential spirit of the present disclosure, the above embodiments are appropriately made and changes fall within the scope of protection of the present disclosure.

What is claimed is:

1. A pre-diagnosis system comprising:
a user interface unit configured to establish an interpretation task and upload first information; wherein the first information is visiting information of a patient; and
an AI interpretation unit configured to receive and execute the interpretation task, and generate an interpretation result;
a data unit configured to obtain second information from the first information and update the second information; wherein the second information is a valid visiting information in the first information; and
a management unit configured to obtain updated second information and the interpretation result and set visiting state of the patient according to the second information and the interpretation result.

2. The pre-diagnosis system of claim 1, wherein the AI interpretation unit comprises a plurality of AI model programs and a plurality of interpretation modules, when the AI interpretation unit receives the interpretation task, the AI interpretation unit selects a corresponding AI model program according to the interpretation task, and the AI model program is configured to select a corresponding interpretation module to execute the interpretation task.

3. The pre-diagnosis system of claim 1, wherein the data unit comprises a database, and the database is configured to store the first information and the interpretation result.

4. The pre-diagnosis system of claim 3, wherein the data unit further comprises a data collection module, the data collection module is connected to the database, and the data collection module is configured to obtain the second information from the database.

5. The pre-diagnosis system of claim 3, wherein the data unit further comprises a data update module, the data update module is connected to the database, the data update module is configured to update the second information and store the updated second information in the database.

6. The pre-diagnosis system of claim 5, wherein the management unit comprises a management module, the management module is configured to obtain the interpretation result and the updated second information from the data unit, and set the visiting state of the patient according to the interpretation result and the updated second information.

7. The pre-diagnosis system of claim 6, wherein the management unit further comprises a display module, the display module is configured to display a displaying information according to the visiting state set by the management module.

8. The pre-diagnosis system of claim 1, wherein the user interface unit is configured to set a first preset time, when a visiting time limit of the patient has not exceeded the first preset time, the data unit sets the first information of the patient as the second information.

9. The pre-diagnosis system of claim 5, wherein the user interface unit is configured to set a second preset time, the data update module updates the second information according to the second preset time.

10. The pre-diagnosis system of claim 1, wherein the management unit is configured to send the visiting state of the patient to a mobile terminal, and the mobile terminal displays the visiting state of the patient.

11. A pre-diagnosis method comprising:
establishing an interpretation task and uploading first information; wherein the first information is visiting information of a patient; and
receiving and executing the interpretation task, and generate an interpretation result;
obtaining second information from the first information and updating the second information; wherein the second information is a valid visiting information in the first information; and
obtaining updated second information and the interpretation result and setting visiting state of the patient according to the second information and the interpretation result.

12. The pre-diagnosis method of claim 11, further comprising:
selecting a corresponding AI model program according to the interpretation task the interpretation task is received; wherein the AI model program is configured to select a corresponding interpretation module to execute the interpretation task.

13. The pre-diagnosis method of claim 11, further comprising:
storing the first information and the interpretation result.

14. The pre-diagnosis method of claim 13, further comprising:
obtaining the second information from the database.

15. The pre-diagnosis method of claim 13, further comprising:
updating the second information and storing the updated second information in a database.

16. The pre-diagnosis method of claim 15, further comprising:
obtaining the interpretation result and the updated second information and setting the visiting state of the patient according to the interpretation result and the updated second information.

17. The pre-diagnosis method of claim 16, further comprising:
displaying a displaying information according to the visiting state.

18. The pre-diagnosis method of claim 11, further comprising:
setting a first preset time and setting the first information of the patient as the second information when a visiting time limit of the patient has not exceeded the first preset time.

19. The pre-diagnosis method of claim 15, further comprising:
setting a second preset time and updating the second information according to the second preset time.

20. The pre-diagnosis method of claim 11, further comprising:
sending the visiting state of the patient to a mobile terminal;
wherein the mobile terminal displays the visiting state of the patient.
